(19) United States
(12) Patent Application Publication
(10) Pub. No.: US 2026/0151194 A1
(43) Pub. Date: Jun. 4, 2026

(54) NAVIGATION SYSTEM FOR SURGICAL INSTRUMENT AND FASTENING DEVICE

(71) Applicant: Point Robotics Medtech Inc., Hsinchu County (TW)

(72) Inventors: CHAO-WEI WU, Hsinchu County (TW); TING-YUN FANG, Hsinchu County (TW); KUN-PIN HUANG, Hsinchu County (TW)

(21) Appl. No.: 19/084,688

(22) Filed: Mar. 19, 2025

(30) Foreign Application Priority Data
Dec. 2, 2024 (TW) 113146483

(51) Int. Cl. A61B 34/30; A61B 34/20

(52) U.S. Cl. CPC A61B 34/30; A61B 34/20; A61B 2034/2046; A61B 2034/301

(57) ABSTRACT

A navigation system for a surgical instrument, includes a robot arm, a fastening device, and a navigation marker element. The fastening device includes a main body, a receiving portion, a socket member, a first pressing member, and a multi-link mechanism. The receiving portion and the robot arm are connected to the main body. The receiving portion has a receiving hole. The socket member is detachably arranged in the receiving hole and has a through hole that allows a surgical instrument to be inserted into. The socket member is provided with a gap and includes a limiting portion, and the gap communicates with the through hole. The first pressing member is disposed on the main body. The multi-link mechanism is disposed in the main body and the receiving portion, and is connected to the first pressing member and the socket member.

### CROSS-REFERENCE TO RELATED PATENT APPLICATION

This application claims the benefit of priority to Taiwan Patent Application No. 113146483, filed on Dec. 2, 2024. The entire content of the above identified application is incorporated herein by reference.

### FIELD OF THE DISCLOSURE

The present disclosure relates to a navigation system and a fastening device, and more particularly to a navigation system and a fastening device that are capable of simplifying the operating process and securely clamping a surgical instrument.

### BACKGROUND OF THE DISCLOSURE

A spinal fusion operation involves several steps: making a small incision on the patient's skin; extending a trocar or bone drill into the patient body; and then drilling a hole at the pedicel for inserting a guide wire through the hole. Afterward, a hollow pedicle screw is screwed into the spine along the guide wire to complete implantation of the screw.

In the related art, the implantation of hollow pedicle screws is performed by using a robot arm along with surgical instruments, which require complex surgical steps and multiple replacements of different instruments. During the process of replacing instruments, it is necessary to repeatedly loosen or tighten the bolts on the robot arm, for confirming that the surgical instruments are at appropriate positions. However, existing robot arms are designed to adapt to only a limited number of surgical instruments of different sizes. As a result, it can't be guaranteed that the existing robot arms may reliably clamp surgical instruments of various sizes, and the clamping force of the existing robot arms can't be properly adjusted to allow free movement of the surgical instruments. Furthermore, the existing robot arms and surgical instruments are generally equipped with a plurality of navigation marker elements. During the surgery, some of the navigation marker elements may be inadvertently obstructed by the doctor, thereby resulting in interference to operation of the surgical instrument navigation system.

### SUMMARY OF THE DISCLOSURE

In response to the above-referenced technical inadequacies, the present disclosure provides a navigation system for a surgical instrument and a fastening device that are capable of simplifying the operating process and securely clamping the surgical instrument.

In order to solve the above-mentioned problems, one of the technical aspects adopted by the present disclosure is to provide a fastening device, which includes a main body, a receiving portion, a socket member, a first pressing member, and a multi-link mechanism. One end of the main body is connected to a robot arm, and the receiving portion is in the main body. The receiving portion has a receiving hole. The socket member is detachably disposed within the receiving hole. The socket member has a through hole that allows a surgical instrument to be inserted into. A side of the socket member is provided with a gap along an axial direction of the through hole, and the gap communicates with the through hole. The socket member includes a limiting portion that is disposed beside the gap. The first pressing member is disposed on the main body. The multi-link mechanism is disposed in the main body and the receiving portion, and connected to the first pressing member and the socket member. The first pressing member can be pressed for driving the multi-link mechanism to push against the limiting portion to narrow the gap, such that the socket member is compressed to clamp the surgical instrument.

In order to solve the above-mentioned problems, another one of the technical aspects adopted by the present disclosure is to provide a navigation system for a surgical instrument, which includes a robot arm, a fastening device, and a navigation marker element. The fastening device is connected to the robot arm. The fastening device is used to clamp the surgical instrument. The navigation marker element is disposed on the surgical instrument.

Therefore, in the navigation system and the fastening device provided by the present disclosure, through the design of the gap in the socket member, the socket member can be deformed by applied force. When the socket member is installed on the robot arm, the mechanical structure in the robot arm compresses and deforms the socket member to clamp the surgical instrument. Furthermore, the fastening device provided by the present disclosure can control the force for clamping the surgical instruments of different sizes by adjusting the deformation extent of the socket member. Therefore, the fastening device can not only securely clamp the surgical instrument, but also can be appropriately loosened to allow the surgical instrument to move freely. In addition, in the navigation system provided by the present disclosure, the robot arm and the surgical instrument share a navigation marker element, which simplifies the calibration process of the robot arm and reduces the time required.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a fastening device clamping a surgical instrument according to an embodiment of the present disclosure;

FIG. 2 is a schematic view of the fastening device according to the embodiment of the present disclosure;

FIG. 3 is a schematic exploded view of the fastening device according to the embodiment of the present disclosure;

FIG. 4 is a schematic top view of a socket member according to the embodiment of the present disclosure;

FIG. 5 is a schematic side view of a cover according to the embodiment of the present disclosure;

FIG. 6 is a schematic view of a first pressing member, a second pressing member, a multi-link mechanism, an outer ring portion, and the socket member according to the embodiment of the present disclosure;

FIG. 7 is a schematic view of a third pressing member, the outer ring portion, and the socket member according to the embodiment of the present disclosure;

FIG. 8 is a schematic cross-sectional view of the fastening device in an unlocked state according to the embodiment of the present disclosure;

FIG. 9 is a schematic cross-sectional view of the fastening device in a locked state according to the embodiment of the present disclosure;

FIG. 10 is a schematic view of a surgical instrument navigation system according to the embodiment of the present disclosure;

FIG. 11 is a schematic exploded view of the surgical instrument, the socket member, and the fastening device according to the embodiment of the present disclosure;

FIG. 12 is a schematic view of a guide wire passing through the surgical instrument that is fixed on the fastening device according to the embodiment of the present disclosure;

FIG. 13 is a schematic view of the guide wire being left in the fastening device according to the embodiment of the present disclosure;

FIG. 14 is a schematic view of another surgical instrument being inserted into the fastening device along the guide wire according to the embodiment of the present disclosure; and

FIG. 15 is a schematic view of the guide wire being removed from the fastening device according to the embodiment of the present disclosure.

### DETAILED DESCRIPTION OF THE EXEMPLARY EMBODIMENTS

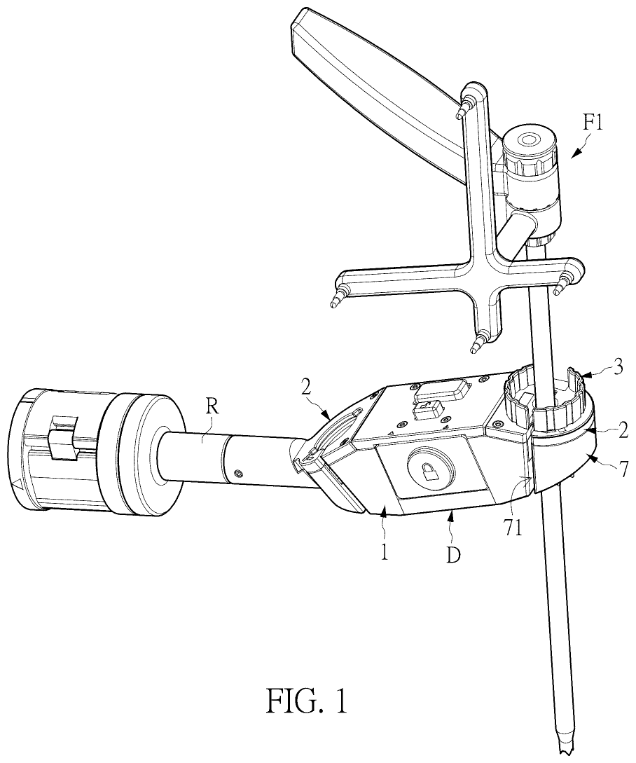

Reference is made to FIG. 1 to FIG. 3. The present disclosure provides a fastening device D that is used for clamping a surgical instrument F1. The fastening device D includes a main body 1, a receiving portion 2, a socket member 3, a first pressing member 4, a multi-link mechanism 5, and a second pressing member 6.

The main body 1 is connected to a robot arm R. For example, the fastening device D can include two receiving portions 2, and the two receiving portions 2 respectively locate near two sides of the main body 1. The receiving portion 2 has a receiving hole 20. The socket member 3 has a through hole 30. The socket member 3 is detachably disposed in the receiving hole 20. The surgical instrument F1 can pass through the through hole 30 of the socket member 3 and is fixed on the socket member 3. The first pressing member 4 and the second pressing member 6 extend out from the main body 1. The first pressing member 4 and the second pressing member 6 are used to control a clamping force of the socket member 3 to the surgical instrument F1. Specifically, most of the components of the first pressing member 4 and the second pressing member 6 are located inside the main body 1, and only pressing portions 43, 62 of the first and second pressing members 4, 6 are exposed to outside of the main body 1. The multi-link mechanism 5 is disposed between the main body 1 and the receiving portion 2, and is connected to the first pressing member 4 and the socket member 3.

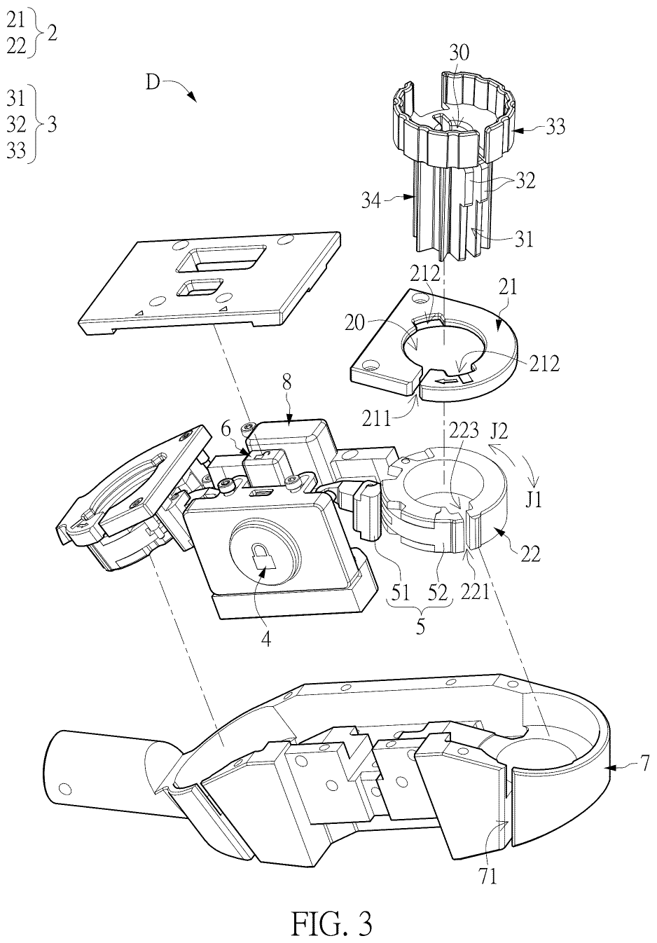

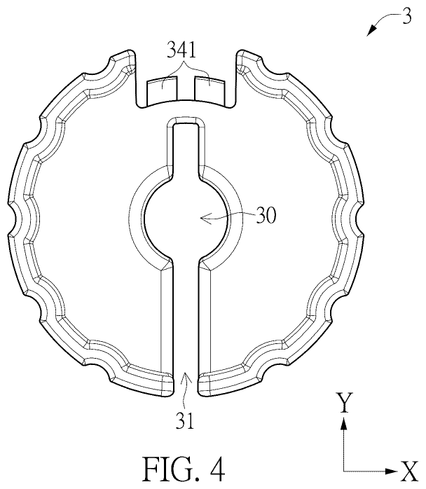

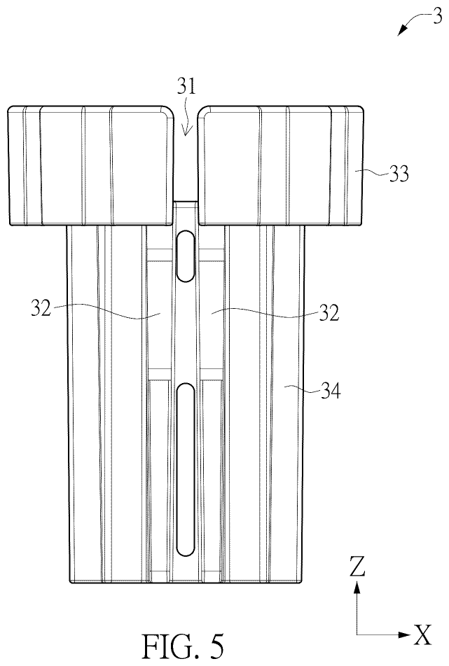

Reference is made to FIG. 3 to FIG. 5. A side of the socket member 3 has a gap 31. The gap 31 extends along an axial direction (i.e., a Z-axis direction) of the through hole 30. Moreover, the gap 31 further extends along a Y-axis direction and communicates with the through hole 30. The socket member 3 includes two limiting portions 32, and the two limiting portions 32 are respectively located at two edges of the gap 31 and extend along the edges of the gap 31. The socket member 3 further includes two limiting protrusions 341, and the two limiting protrusions 341 are located opposite to the gap 31. The shape of the socket member 3 is a T-shape cylinder. The socket member 3 includes a head portion 33 and a body portion 34. A diameter of the head portion 33 is greater than a diameter of the body portion 34. The through hole 30 and the gap 31 extend from the head portion 33 to the body portion 34 along the axial direction. The limiting portions 32 and the limiting protrusions 341 are disposed at opposite sides of the body portion 34.

As shown in FIG. 3, the receiving portion 2 includes a cover 21 and an outer ring portion 22. The cover 21 has a first notch 211 and two limiting grooves 212 that are opposite to each other. The first notch 211 and the two limiting grooves 212 are disposed along a periphery of the receiving hole 20. The outer ring portion 22 has a second notch 221 that is disposed along the periphery of the receiving hole 20. At a position of the inner side of the outer ring portion 22, which corresponds to the second notch 221, the outer ring 22 includes a recess 223. The fastening device D further includes an outer frame 7. The outer frame 7 forms the main body 1 and is connected to the receiving portion 2. The socket member 3, the first pressing member 4, the multi-link mechanism 5, and the second pressing member 6 are disposed inside the outer frame 7. The outer frame 7 includes a third notch 71. The cover 21 is fixed to the outer frame 7. The outer ring portion 22 is detachably disposed inside the outer frame 7 and is located below the cover 21.

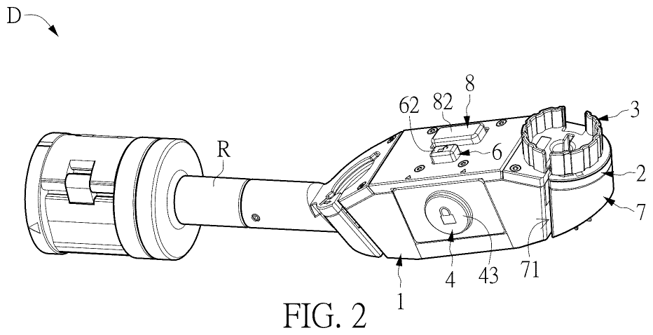

The socket member 3 is a disposable accessory which is replaceable. The socket member 3 can be, for example, a deformable plastic cup. Different socket members 3 may have through holes 30 of different sizes, and the socket members 3 may adapt to different surgical instruments. The socket member 3 is required not only to be able to be assembled in the receiving portion 2, but also be detached from the receiving portion 2. Reference is made to FIGS. 2, 3, and 6. When the socket member 3 is assembled to the receiving portion 2, the limiting portions 32 and the limiting protrusions 341 align with and pass through the two limiting grooves 212, respectively. Therefore, the limiting portions 32 are disposed in the recess 223, and the gap 31 aligns with the second notch 221 (shown in FIG. 3). In other words, before installing the socket member 3 into the receiving portion 2, the second notch 221 needs to be aligned with one of the two limiting grooves 212.

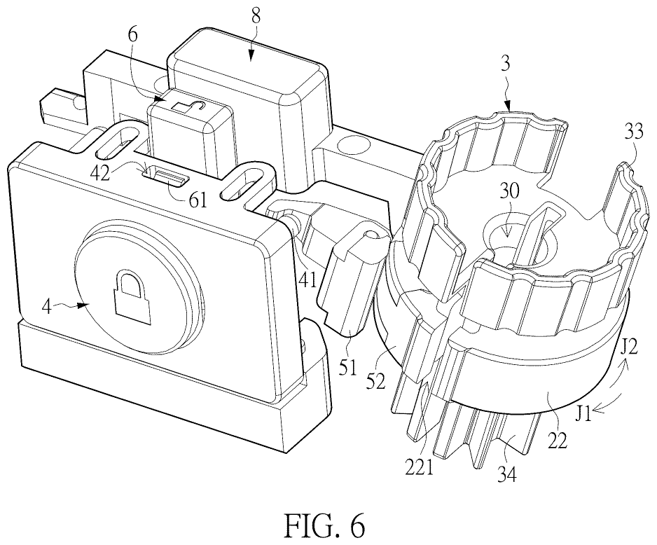

When the socket member 3 is installed in the receiving portion 2, the socket member 3 can be rotated to drive the outer ring portion 22 to rotate relative to the cover 21 along a first direction J1 (shown in FIG. 6, where the cover 21 is omitted from the receiving portion 2 for clarity of illustration), aligning the gap 31 and the second notch 221 with the first notch 211 and the third notch 71. In the meantime, the limiting portions 32 and the limiting protrusions 341 no longer align with the two limiting grooves 212 but misalign with them. Therefore, movement of the socket member 3 along the axial direction (i.e., the Z-axis direction) is limited. Consequently, the socket member 3 cannot be pulled out from the receiving hole 20 because the limiting portions 32 get stuck by the cover 21. In other words, the socket member 3 is fixed in the receiving portion 2 and will not fall out.

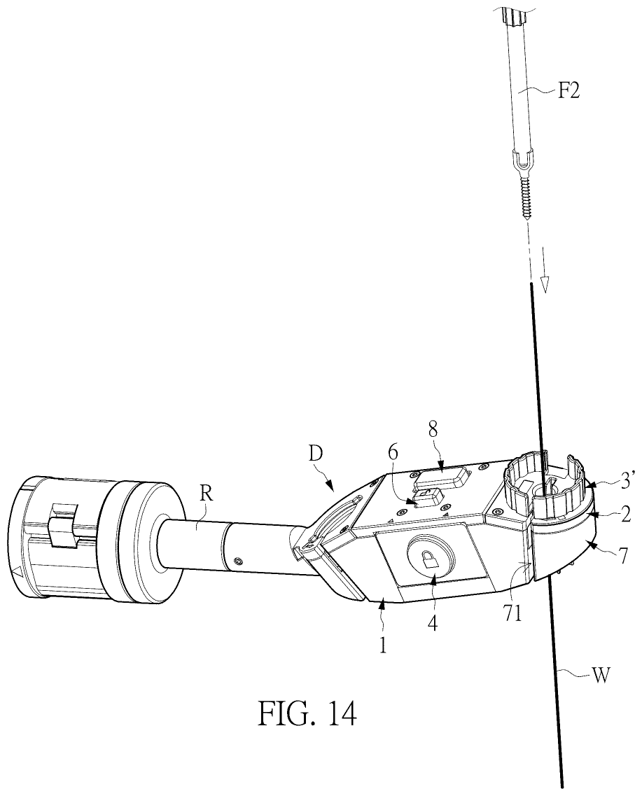

Additionally, it should be noted that the gap 31, the first notch 211, and the second notch 221 align with each other to form a channel, and objects placed within the socket member 3, such as a guide wire W (which will be mentioned later, shown in FIG. 14) can be retrieved through the channel.

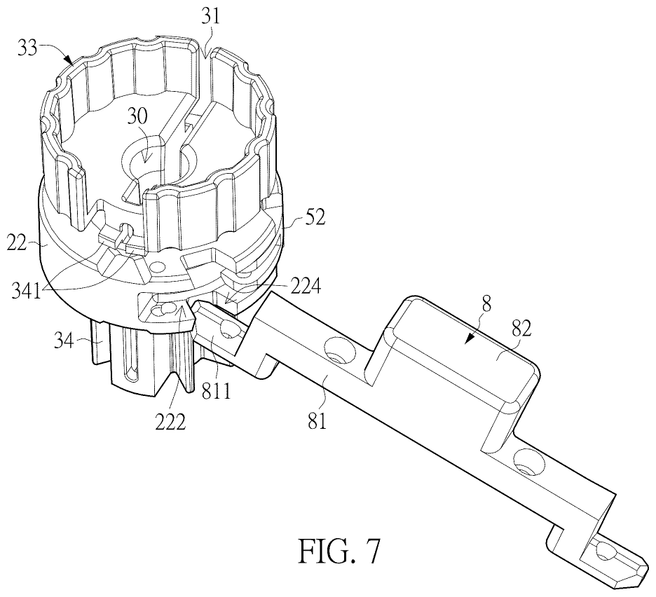

Reference is made to FIGS. 3, 6, and 7. The fastening device D further includes a third pressing member 8. For clarity of illustration, the first pressing member 4 and the second pressing member 6 are omitted in FIG. 7. The third pressing member 8 includes a rod structure 81 and a pressing portion 82. The rod structure 81 is disposed in the main body 1, while the pressing portion 82 is exposed to outside of the main body 1. When the socket member 3 is fixed in the receiving hole 20, a convex portion 811 of the third pressing member 8 extends into the receiving portion 2 and is engaged with a concave portion 224 of the outer ring portion 22 (shown in FIG. 7), thereby limiting the rotation of the outer ring portion 22.

On the other hand, if a user wants to detach the socket member 3 from the receiving portion 2, he can push the third pressing member 8 to move the rod structure 81 downward and cause the convex portion 811 to be disengaged from the concave portion 224, thereby releasing the limitation on the rotation of the outer ring portion 22. Then, the socket member 3 can be rotated again, causing the outer ring portion 22 to rotate relative to the cover 21 along a second direction J2 that is opposite to the first direction J1, aligning the gap 31 and the second notch 221 with one of the limiting grooves 212. As such, the socket member 3 can be detached from the receiving portion 2.

Reference is further made to FIGS. 6 to 8. The multi-link mechanism 5 includes a connecting rod 51 and a sliding block 52 that are connected to each other. The connecting rod 51 is disposed in the main body 1, while the sliding block 52 is disposed in the receiving portion 2. The outer ring portion 22 further includes a sliding groove 222, and the sliding block 52 is movably disposed in the sliding groove 222. The first pressing member 4 includes a protruding column 41 and at least one retaining groove 42. The protruding column 41 abuts against the connecting rod 51, and the sliding block 52 abuts against one of the limiting portions 32. The second pressing member 6 includes at least one stopping portion 61.

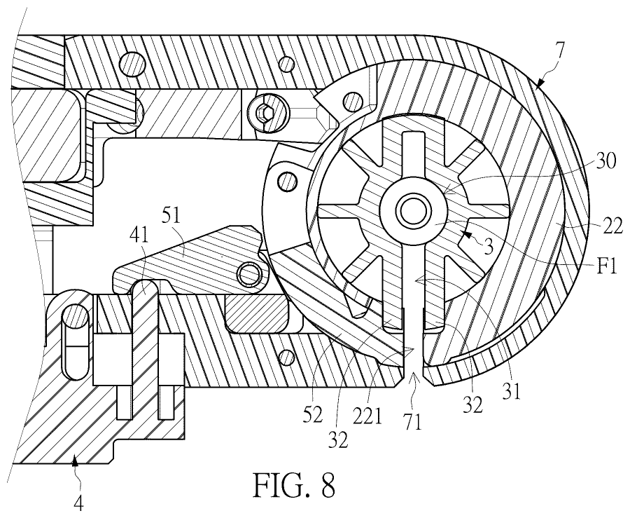

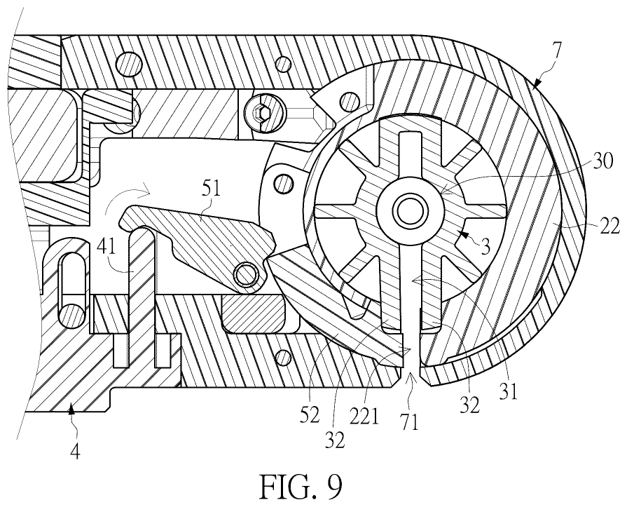

By operating the first pressing member 4 and the second pressing member 6, the socket member 3 can be switched between an unlocked state and a locked state. Reference is made to FIGS. 6, 8, and 9. FIG. 8 shows the socket member 3 in the unlocked state, while FIG. 9 shows the socket member 3 in the locked state. When the first pressing member 4 is pressed (shown in FIGS. 6 and 9), the protruding column 41 pushes the connecting rod 51 forward, driving the sliding block 52 to push one of the limiting portions 32 closer to the other of the limiting portions 32. As a result, the gap 31 between the two limiting portions 32 is narrowed, causing the socket member 3 to deform and generate the clamping force to clamp the surgical instrument F1. Furthermore, as the first pressing member 4 is pressed, the first pressing member 4 moves closer to the second pressing member 6, and the at least one stopping portion 61 then engages with the at least one retaining groove 42.

It should be noted that as the first pressing member 4 is pressed, the first pressing member 4 abuts against a spring element (not shown in the figures). In contrast, when the first pressing member 4 is released, an elastic force generated by the spring element pushes the first pressing member 4 backward to its original position. Therefore, through the design that the at least one stopping portion 61 engages with the at least one retaining groove 42, the position of the first pressing member 4 can be limited and maintained in a pressed position.

On the other hand, when the second pressing member 6 is pressed, the at least one stopping portion 61 is disengaged from the at least one retaining groove 42. Without the limitation imposed by the at least one stopping portion 61, the first pressing member 4 returns from the pressed position shown in FIG. 9 to an unpressed position shown in FIG. 8 due to the elastic force of the spring element. The multi-link mechanism 5 no longer moves jointly due to restoration of the first pressing member 4. That means, the protruding column 41 retracts and no longer pushes the connecting rod 51, so that the connecting rod 51 does not push the sliding block 52 to compress the limiting portions 32 of the socket member 3. Therefore, the gap 31 between the two limiting portions 32 returns to its original width, allowing the socket member 3 to recover its original dimension and release clamping to the surgical instrument F1, which enables the surgical instrument F1 to adjust its position or orientation, or to be removed from the socket member 3.

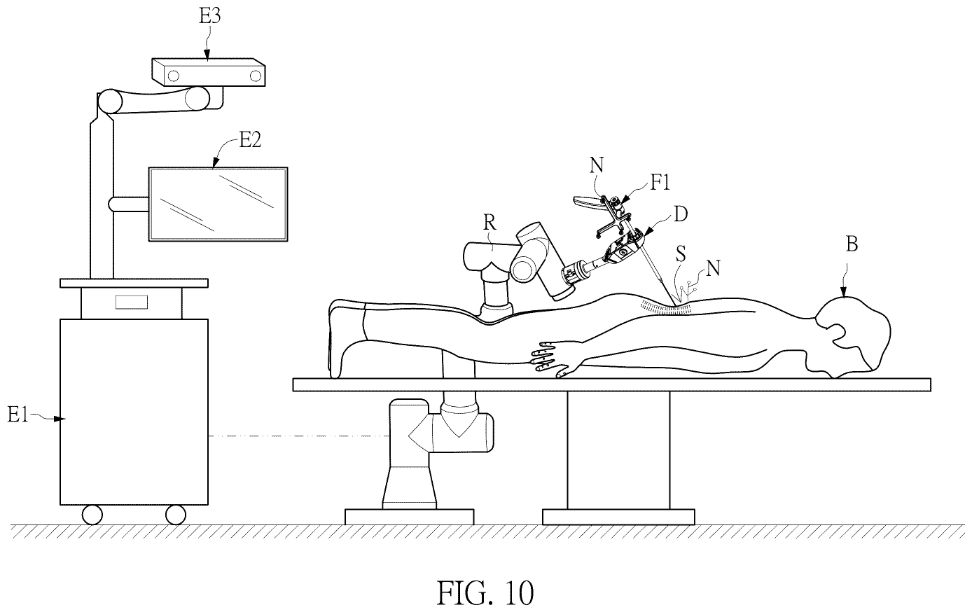

Reference is made to FIG. 10. The fastening device D can be applied in a navigation system for a surgical instrument. Specifically, the navigation system includes a robot arm R, the fastening device D connects with the robot arm R. The fastening device D clamps the surgical instrument F1, and a plurality of navigation marker elements N are distributed on the surgical instrument F1 and near a surgical site S of a patient B. For example, the surgical site S can be the spinal region of the patient B. The navigation marker elements N include a dynamic reference frame (DRF) and a plurality of optical elements mounted on the dynamic reference frame. The optical elements can be reflective balls or marker elements that generate perceptible signals.

Additionally, the navigation system further includes a processing device E1, a display device E2, and an optical tracker E3. The optical tracker E3 and the display device E2 are electrically connected to the processing device E1. The plurality of navigation marker elements N can serve as spatial positioning reference points for establishing a spatial coordinate system, while the optical tracker E3 can sense, detect, and record the coordinates of the plurality of optical elements on the navigation marker elements N, sending this information (i.e., the coordinates of the plurality of optical elements) to the processing device E1 for appropriate calculation and/or storage. Consequently, the processing device E1 acquires images near the surgical site S and integrates previously obtained medical images, such as computed tomography (CT) or magnetic resonance imaging (MRI), to create a three-dimensional anatomy model of the area surrounding the surgical site S. The completed three-dimensional anatomy model can be displayed on a navigation interface of the display device E2.

It should be noted that the robot arm R does not have additional navigation marker elements N but shares one set of navigation marker elements N on the surgical instrument F1. In existing technology, each of a conventional robot arm and a conventional surgical instrument is generally equipped with an individual set of navigation marker elements. Therefore, during surgery, some of the navigation marker elements may easily be obstructed by the doctor, such that the optical tracker can't track the position of the conventional surgical instrument, thereby interfering with operation of the conventional navigation system. Furthermore, if the navigation marker elements on the conventional robot arm are obstructed during the surgery, the correct position of the conventional robot arm can't be displayed on a navigation interface. Additionally, there may be some deviations when detecting the navigation marker elements on the conventional robot arm and on the conventional surgical instrument, leading to a situation that the conventional surgical instrument and the conventional robot arm seem to be non-coaxial on the navigation interface even though they are mechanically coaxial.

Therefore, in the present disclosure, the robot arm R and the surgical instrument F1 share one set of navigation marker elements N and are calibrated through adjustments to the mechanical parameters of the robot arm R. Since there are no additional navigation marker elements on the robot arm R, the problem of obstructing some of the navigation marker elements in existing technology can be avoided. Moreover, sharing one set of navigation marker elements N between the robot arm R and the surgical instrument F1 simplifies the calibration process and saves time for calibrating the robot arm R, and the issue that the positions of the conventional robot arm and the conventional surgical instrument can't be correctly displayed on the navigation interface is resolved.

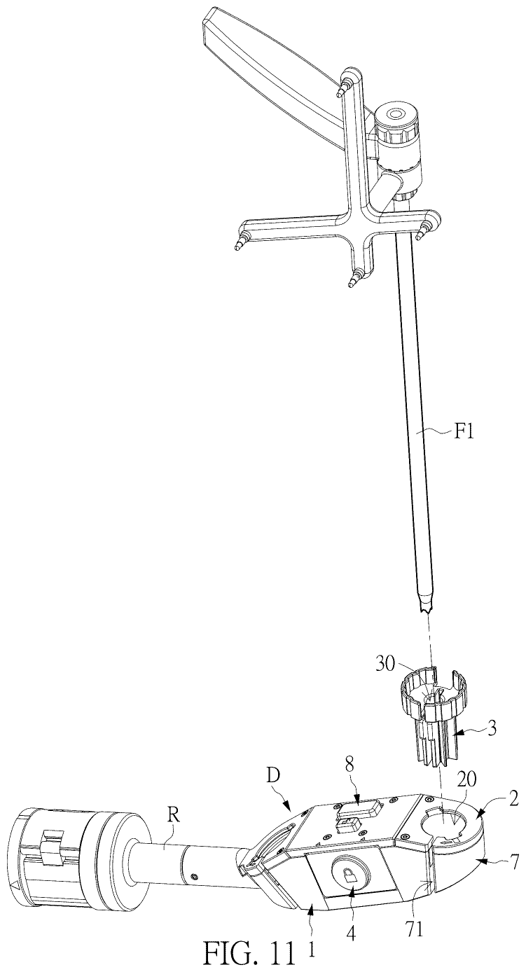

Reference is made to FIGS. 10 to 15. The surgical instrument F1 can be, for example, a trocar. The surgical instrument F1 is disposed in the fastening device D of the robot arm R, and the first pressing member 4 is pressed to allow the fastening device D to clamp the surgical instrument F1 (shown in FIGS. 10 and 11). Through the navigation interface generated by the navigation system, the surgical instrument F1 creates the correct surgical path at the surgical site S (not shown in the figures), allowing the user to insert a drill bit through the trocar and drill a guide hole at the surgical site S. Additionally, the socket member 3 is selected to fit the size of the surgical instrument F1.

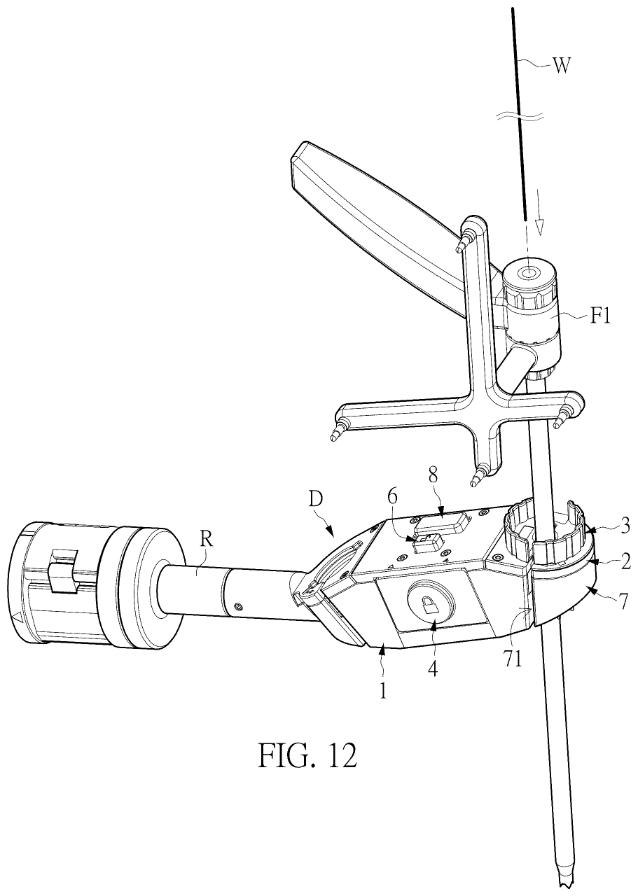

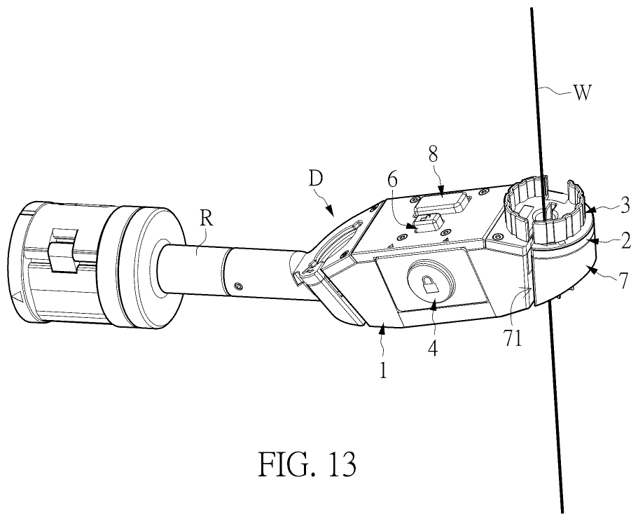

Then, a guide wire W is inserted to the guide hole through the surgical instrument F1 (shown in FIG. 12), and the second pressing member 6 is pressed to release the fastening device D, allowing the surgical instrument F1 to be detached and leaving only the guide wire W in the receiving portion 2 (shown in FIG. 13). After that, the third pressing member 8 is pressed to release the socket member 3, allowing it to be removed, and another socket member 3′ with different size is selected and installed in the receiving portion 2. Subsequently, another surgical instrument F2, such as a screw implantation device with a hollow pedicle screw, is disposed in the socket member 3′ which is located in the receiving portion 2. The socket member 3′ is selected to fit the size of the surgical instrument F2. Then, the first pressing member 4 is pressed again to hold the surgical instrument F2, and the surgical instrument F2 is enabled to freely move up or down. After the surgical instrument F2 is installed in the socket member 3′, the hollow pedicle screw can be rotated for screwing into the surgical site S (i.e., the spinal region) along the guide wire W (shown in FIG. 14), thereby completing the implantation of the hollow pedicle screw.

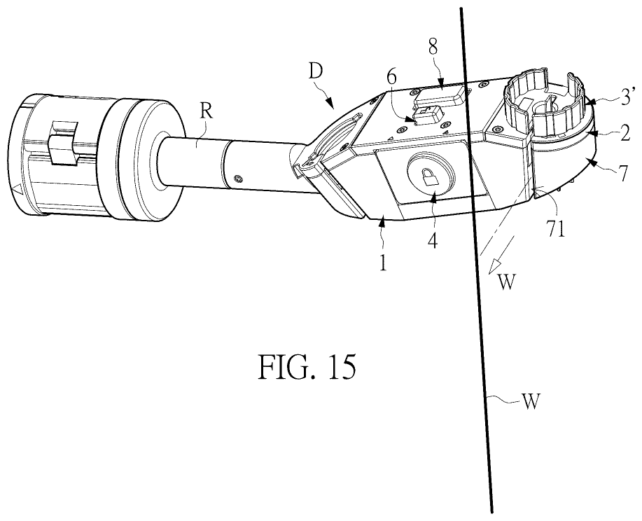

Afterwards, the robot arm R is moved away from the surgical site S, and the surgical instrument F2 and the guide wire W are simultaneously removed. Specifically, the second pressing member 6 is pressed again to release holding of the surgical instrument F2 by the fastening device D, allowing the surgical instrument F2 to be removed while the guide wire W can be drawn out through the third notch 71 (shown in FIG. 15).

### Beneficial Effects of the Embodiment

In the disclosed fastening device D, through the design of the gaps 31 in the socket members 3 and 3′, the socket members 3 and 3′ may tolerate a certain amount of deformation. Different socket members 3 and 3′ may have through holes 30 of different sizes which adapt to different surgical instruments F1 and F2. When the socket members 3 and 3′ are disposed at the robot arm, the structural design of the fastening device D is used to squeeze and deform the socket members 3 and 3′, to securely clamp the surgical instrument F1 and hold the surgical instrument F2. Furthermore, in the disclosed fastening device D, the force for clamping/holding the surgical instruments F1, F2 with different sizes can be controlled by compressing the socket member 3, 3′. Therefore, the fastening device D can not only securely clamp the surgical instrument F1 and hold the surgical instrument F2, but also can be appropriately loosened for the user to freely remove the surgical instruments F1 and F2.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A fastening device, comprising:
a main body, wherein one end of the main body is connected to a robot arm;
a receiving portion, wherein the receiving portion is in the main body, and the receiving portion has a receiving hole;
a socket member detachably disposed within the receiving hole, wherein the socket member has a through hole that allows a surgical instrument to be inserted into, a side of the socket member is provided with a gap along an axial direction of the through hole, the gap communicates with the through hole, and the socket member includes a limiting portion that is disposed beside the gap;
a first pressing member disposed on the main body; and
a multi-link mechanism disposed in the main body and the receiving portion, wherein the multi-link mechanism is connected to the first pressing member and the socket member;
wherein the first pressing member can be pressed for driving the multi-link mechanism to push against the limiting portion to narrow the gap, such that the socket member is compressed to clamp the surgical instrument.

2. The fastening device according to claim 1, wherein the multi-link mechanism includes a connecting rod and a sliding block, the connecting rod is disposed in the main body, the sliding block is disposed in the receiving portion, the first pressing member includes a protruding column, the protruding column abuts against the connecting rod, and the sliding block abuts against the limiting portion.

3. The fastening device according to claim 2, further comprising a second pressing member, wherein the second pressing member is disposed on the main body, the second pressing member includes at least one stopping portion, and the first pressing member further includes at least one retaining groove; wherein, when the first pressing member is pressed, the at least one stopping portion engages with the at least one retaining groove to limit the first pressing member, and the protruding column pushes against the connecting rod and drives the sliding block to push the limiting portion, such that the socket member is compressed to clamp the surgical instrument.

4. The fastening device according to claim 3, wherein, when the second pressing member is pressed, the at least one stopping portion disengages from the at least one retaining groove to return the first pressing member, and the multi-link mechanism stops compressing the socket member due to return of the first pressing member, such that the socket member is restored and the surgical instrument is no longer clamped.

5. The fastening device according to claim 2, wherein the receiving portion includes a cover and an outer ring portion, the outer ring portion is movably disposed below the cover, the cover includes a first notch and two limiting grooves that are opposite to each other, the first notch and the two limiting grooves are disposed along an edge of the receiving hole, and the outer ring portion includes a second notch that is disposed along the edge of the receiving hole.

6. The fastening device according to claim 5, wherein the outer ring portion includes a sliding groove, the sliding block is moveably disposed in the sliding groove and located at an edge of the second notch, and an inner side of the outer ring portion is provided with a recess that corresponds to the second notch; wherein, when the socket member is disposed within the receiving hole, the limiting portion is disposed in the recess.

7. The fastening device according to claim 5, wherein a shape of the socket member is a T-shape cylinder, the socket member includes a head portion and a body portion, a diameter of the head portion is greater than a diameter of the body portion, the limiting portion is disposed on the body portion, the gap extends from the head portion to the body portion along the axial direction of the through hole, and the body portion is provided with a limiting protrusion on an opposite side of the gap.

8. The fastening device according to claim 7, wherein the socket is disposed within the receiving hole, the limiting portion and the limiting protrusion align with and pass through the two limiting grooves, respectively, and the gap aligns with the second notch; wherein, after the socket member is disposed within the receiving hole, the socket member is rotated to drive the outer ring portion to rotate in a first direction relative to the cover, such that the gap and the second notch align with the first notch, the limiting portion and the limiting protrusion are misaligned with the two limiting grooves, and motion of the socket member along the axial direction is thus limited.

9. The fastening device according to claim 8, further comprising an outer frame, wherein the outer frame covers the outer ring portion, the outer frame has a third notch, and the third notch aligns with the first notch and the second notch.

10. The fastening device according to claim 8, further comprising a third pressing member that is disposed on the main body; wherein, when the socket member is fixed within the receiving hole, a convex portion of the third pressing member extends to the receiving portion and is engaged with a concave portion of the outer ring portion, such that rotation of the outer ring portion is limited.

11. The fastening device according to claim 10, wherein the third pressing member is pressed to disengage the convex portion from the concave portion, the socket member is rotated to drive the outer ring portion to rotate in a second direction relative to the cover, such that the limiting portion and the limiting protrusion respectively align with the two limiting grooves to release limitation to the position of the socket member along the axial direction; wherein the second direction is opposite to the first direction.

12. A navigation system for a surgical instrument, comprising:
a robot arm;
the fastening device as claimed in claim 1, the fastening device being connected to the robot arm, wherein the fastening device is used to clamp the surgical instrument; and
a set of navigation marker elements disposed on the surgical instrument.
